(19) United States
(12) Patent Application Publication
(10) Pub. No.: US 2007/0003945 A1
(43) Pub. Date: Jan. 4, 2007

(54) Method for manufacturing a biosensor element and for testing the same

(76) Inventors: Miwako Nakahara, Tokyo (JP); Takashi Inoue, Yokohama (JP); Tomonori Saeki, Yokosuka (JP); Osamu Kogi, Yokohama (JP); Noriko Ban, Fujisawa (JP)

Correspondence Address: MATTINGLY, STANGER, MALUR & BRUNDIDGE, P.C., 1800 DIAGONAL ROAD, SUITE 370, ALEXANDRIA, VA, 22314, US

(21) Appl. No.: 11/270,612

(22) Filed: Nov. 10, 2005

(30) Foreign Application Priority Data
Jul. 1, 2005 (JP) 2005-193786

(51) Int. Cl. C12Q 1/68; G01N 33/53; C12M 1/34; G06F 19/00

(52) U.S. Cl. 435/6; 435/7.1; 435/287.2; 702/19

(57) ABSTRACT

When a biomolecule and a biochemical reactant are detected, a white interference method is used to conduct a noncontact and nondestructive detection, and further to conduct efficient and accurate detection. This method is applied to a biosensor element, whereby non-labeled and noncontact quality control can be achieved.

### BACKGROUND OF THE INVENTION

The present invention relates to a method for nondestructive/noncontact testing of a biosensor element on the surface of which a nucleic acid, protein, and the like are immobilized for sensing of biomolecules and chemical reactions, and a method for manufacturing this biosensor element.

The human genome sequence has been entirely deciphered by the human genome project, and currently a subject matter of the study is shifting from the conventional “sequence analysis” to “functional analysis” that examines functions thereof. Data obtained from this functional analysis are considered to be able to provide significant information to elucidate life phenomena, and it is expected that such data may become a key to solve problems in every field associated with living things, such as medical practice, environment, and foods.

In the functional analysis above, it is demanded that a gene having an enormous amount of information and a protein made from the gene be analyzed exhaustively and rapidly. Considering this situation, a biochip has been developed, which is a kind of biosensor element, typified by DNA microarrays and protein chips.

A primary detecting method using the biochip is to attach a fluorescence label to a biomolecule which is to be detected, excite the fluorescence label by a laser, and then detect an emitted fluorescence. In this method, it is needed to attach the fluorescence molecule onto the biomolecule to be detected. When the biomolecule is a protein, there is a concern that the structure of the protein may be drastically changed due to the attachment of the fluorescence molecule.

There is another concern that the fluorescence label may interfere with the reaction between the chip and the biomolecule. It may be difficult to accurately estimate the amount of the biomolecules, due to a yield difference in introducing the fluorescence labels, quantum yield or time course of the fluorescence labels, and variations in sensitivity of the fluorescence detecting system. There is an example of another detecting method, which employs surface plasmon resonance (SPR) (U.S. Pat. No. 6,207,381, referred to as “patent document 1”).

However, in order to detect a biomolecule within a microarea, which is captured on the biochip, it is difficult to use SPR for detecting the biochip under present circumstances, since the space resolving power of SPR is low. Therefore, a detecting method which is capable of detecting a biomolecule without using the label, and also capable of detecting the microarea, has been demanded.

Here, a general method for manufacturing the biochip will be explained. There are mainly two methods to manufacture the biochip. One is a method in which an amino acid and a nucleic acid base are sequentially immobilized one by one on a substrate, by means of photolithography or ink-jet, whereby probe biomolecules such as proteins or DNAs (deoxyribonucleic acids) are synthesized on the substrate in-situ (see U.S. Pat. No. 5,424,186, referred to as “patent document 2”). The other is a method in which the probe biomolecules are synthesized ex-situ, and subsequently immobilized on the substrate (U.S. Pat. No. 5,700,637, referred to as “patent document 3”).

It is expected that the biochip will be used in the future, for example, in medical diagnosis such as diagnosing cancer. If the biochip is used in medical diagnosis, it is necessary that data obtained from the biochip have a high quantitativity and reproducibility. In this case, it is significant to grasp a volume and structure of the probe molecules immobilized on the biochip surface, so as to conduct a quality control.

However, in many cases, the probe biomolecules on the biochip surface are coated with a monolayer, and a film thickness of the film coating the probe biomolecules is in an angstrom order. In addition, the size of the area on which one type of probe DNA is immobilized is in submillimeter order. In order to know a volume and a structure of the probe biomolecule which is subjected to ultra-thin coating on the microarea, it is necessary to have an analyzing technique with an extremely high sensitivity and a sufficient space resolving ability.

### SUMMARY OF THE INVENTION

The problems described above can be solved by providing a non-labeled/non-destructive detecting method as the following. That is, when a biomolecule is detected by a biosensor element, according to this method, the biosensor element is capable of detecting a biomolecule in a non-destructive manner, without labeling in advance the biomolecule to be detected. The problems above can also be solved by providing an analyzing method which is capable of evaluating a volume and structure of the probe biomolecules with high sensitivity and in a simple manner, the probe biomolecules being immobilized in a microarea on the sensor surface, in order to conduct a quality control of the biosensor element.

A mode of a detecting method of a biosensor element according to the present invention has a process for detecting a biomolecule, by use of the biosensor element having probe biomolecules immobilized on a substrate, including,

1) a step which mounts on a stage, a biosensor element where the probe biomolecules are immobilized,

2) a step which irradiates the biosensor element with a white light,

3) a step which detects an interference fringe generated by allowing a reflected light from the biosensor element to interfere with a reflected light from a reference plane,

4) a step which obtains either of a distance and an optical path length between the biosensor element and a source of the white light, either of which maximizes a modulation amount of the interference fringe,

5) a step which calculates a three-dimensional shape of the surface of the biosensor element, from either of the distance and the optical path length,

6) a step which obtains a height T1 of a part where the probe biomolecules are immobilized, from the three-dimensional shape thus calculated,

7) a step which allows the biosensor element to react with a solution containing a target biomolecule,

8) a step which performs all of the steps 1) to 5) as described above, for the biosensor element which has been subjected to the reaction with the target molecule,

9) a step which obtains a height T2 of a part where the probe biomolecules are immobilized, from the three-dimensional shape thus calculated, and

10) a step which calculates a difference (T2−T1), between T2 obtained in step 9) and T1 obtained in step 6).

A mode of a method for manufacturing a biosensor element according to the present invention, having probe biomolecules immobilized on a substrate, includes,

1) a step which mounts on a stage, either of a biosensor element having the probe biomolecules being immobilized, and a biosensor substrate in a state prior to having the probe biomolecules being immobilized,

2) a step which irradiates either of the biosensor element and the substrate with a white light,

3) a step which detects an interference fringe generated by allowing a reflected light from either of the biosensor element and the substrate to interfere with a reflected light from a reference plane,

4) a step which obtains either of a distance and an optical path length between either of the biosensor element and the substrate, and a source of the white light, the distance maximizing a modulation amount of the interference fringe,

5) a step which calculates a three-dimensional shape of the surface of either of the biosensor element and the substrate, from either of the distance and the optical path length,

6) a step which obtains an average height T1 on a part where the probe biomolecules are immobilized, a height variation Cv1, or a surface variation Cv2, and

7) a step which conducts quality control of the probe biomolecules with the thus obtained T1, variation Cv1, and variation Cv2.

According to the present invention, by use of a white light interference method, it is possible to detect a biomolecule and/or a biochemical reaction in a noncontacting and nondestructive manner with the biosensor element. Furthermore, according to the present invention, it is possible to conduct a quality control, by testing the quality of the biosensor element in a noncontacting and nondestructive manner, by use of the above method.

### BRIEF DESCRIPTION OF THE DRAWING

These and other features, objects and advantages of the present invention will become more apparent from the following description when taken in conjunction with the accompanying drawings wherein:

FIG. 1A and FIG. 1B are charts showing a result of a spot shape, film thickness, and film thickness variation, which are obtained by use of the white light.

FIG. 2 is an explanatory diagram showing a procedure to obtain a hybridization amount, by obtaining spot film thicknesses using the white light, before and after the hybridization.

FIG. 3 is a correlation diagram to explain a correlation between the hybridization amount measured by use of the white light and the hybridization amount measured by fluorescence.

### DETAILED DESCRIPTION OF PREFERRED EMBODIMENTS

As a preferred embodiment, an example will be explained, where a detecting method and a manufacturing method according to the present invention have been conducted according to a white light interference method.

A plane type DNA microarray was produced according to a producing method as disclosed in the Japanese Patent laid-open publication No. 2004-28953. Borosilicate glass of a slide glass size was employed as a substrate, and on this substrate, 10,000 types of 50-mer probe DNA were spotted. The diameter of one spot size is around 300 μm, and each probe DNA was immobilized on the substrate on a monolayer level.

This DNA microarray was mounted on an XYZθ stage and immediately below a 10× objective lens. At this timing, the magnification of the lens may be any between 2.5× to 100×. Next, the surface of the microarray is irradiated with a white light from a halogen lamp almost perpendicularly. Then, the reflected light from the top surface of the microarray is allowed to interfere with the reflected light from the reference plane placed in the track of the optical path. This interference light is detected by use of a CCD camera.

Here, scanning by the objective lens is conducted in the z-direction, that is, in the perpendicular direction with respect to the microarray surface. At this timing, when the optical path difference between the reflected light from the microarray top surface and the reflected light from the reference plane becomes zero, the contrast of the interference fringe (modulation amount of the interference fringe) formed by the interference light is maximized.

With respect to each pixel detected by the CCD camera, a distance between the objective lens and the microarray surface is obtained, which maximizes the contrast on each pixel, whereby a three-dimensional shape of the microarray surface can be calculated. In other words, a three-dimensional shape of each spot on which probe DNAs are immobilized can be obtained.

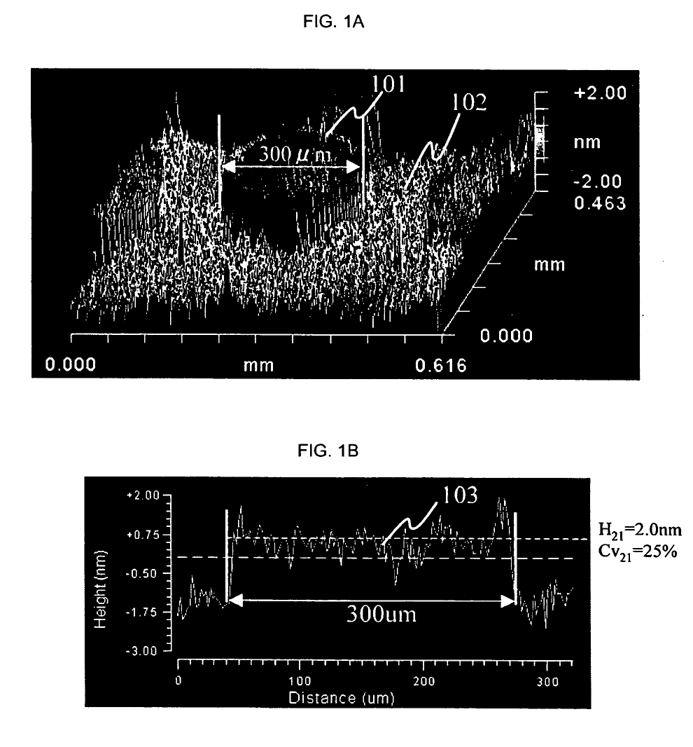

Here, the average film thickness Tn1 within each spot and a value of the height variation Cv (Tn1) (Coefficient of Variation) within each spot are obtained. Here, “n” represents a spot position. FIGS. 1A and 1B show an example of the three-dimensional shape, film thickness, and Cv value of the spot, which are obtained according to the above method. In FIGS. 1A and 1B, numeral 101 is a part where probe DNAs are spotted; numeral 102 is a part where probe DNAs are not spotted; and numeral 103 is a curve which shows a height of a cross section of the probe DNAs. As shown in FIG. 1A, with respect to each spot having a coating of probe DNAs, the film thickness and film thickness variation in the spot can be obtained.

Here, the thus-obtained Tn1 and Cv (Tn1) values are checked against a quality control reference range which is predetermined according to the type of microarray. In other words, T1 as an average value of Tn1, and variations of Tn1, that is, film thickness variations Cv (T1) between spots, are compared with the quality control reference range. If these values are within the quality control reference range, the microarray is determined as an accepted product and handled as a good product. On the other hand, if those values are out of the quality control reference range, the microarray is determined as not accepted, and handled as a defective product.

It is also possible to conduct non-defective/defective judgment with respect to each spot independently. In this case, as to each spot n, if Tn1 and Cv(Tn1) (the film thickness variation in the spot) are within the quality control reference range, this spot n is determined as a favorable spot and will be used for testing. On the other hand, when Tm1 and Cv (Tm1) as to each spot m are out of the quality control reference range, this spot m is determined as a defective spot, and will not be used for detecting an object.

Alternatively, before immobilizing the probe DNAs, a three-dimensional shape of the microarray surface can be obtained according to the same process as described above. Here, the roughness R1 as a coating layer is obtained, which covers the surface in advance for immobilizing the probe DNAs.

This roughness is an Rms value (square mean roughness) obtained from unevenness on the surface that has already been measured, and is expressed as the square root of a mean value as to the square of deviations, from the average line of height to the measured value. This value is checked against the quality control reference range of the roughness which is predetermined according to the type of microarray, and if the obtained value R1 is within the quality control reference range, it is handled as a good product, whereas if it is out of the quality control reference range, it is handled as a defective product. Alternatively, the roughness of the part where the probe DNAs are not spotted, as indicated by numeral 102 in FIG. 1A, is obtained according to the same process as described above, thereby conducting a similar quality control for the coating layer.

Next, the DNA microarray determined as a good product according to the above evaluation was subjected to hybridization reaction with a target DNA which was prepared from the total RNA extracted from a cell in accordance with a method as disclosed in the Japanese Patent laid-open publication No. 2004-28953. Subsequently, the DNA microarray was washed and dried. The DNA microarray subjected to the above processing was mounted again on the aforementioned XYZθ stage, and immediately below the 10× objective lens. The microarray was irradiated with the white light, and the reflected light from the microarray surface was allowed to interfere with the reflected light from the reference plane. The objective lens conducted scanning perpendicular to the microarray, and then a three-dimensional shape of the array surface was obtained.

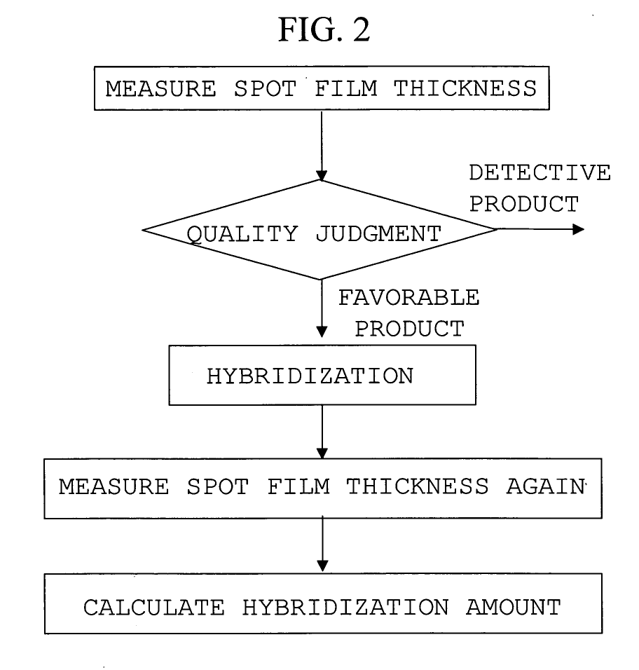

Here, each spot film thickness Tn2 after hybridization is obtained. By subtracting Tn1 obtained before the hybridization on the same spot from the thus-obtained Tn2, the hybridization amount of the target DNA can be obtained with respect to each spot. A series of flow including these quality control processes is shown in FIG. 2.

According to this method above, it is possible to detect the target DNA in a non-labeling manner, without a need to attach a fluorescence label and the like onto the target DNA, and thus a problem in quantitative analysis, such as color fading, can be solved. Furthermore, since the detection and testing can be conducted in a non-contact manner, it is possible to avoid damage against the microarray.

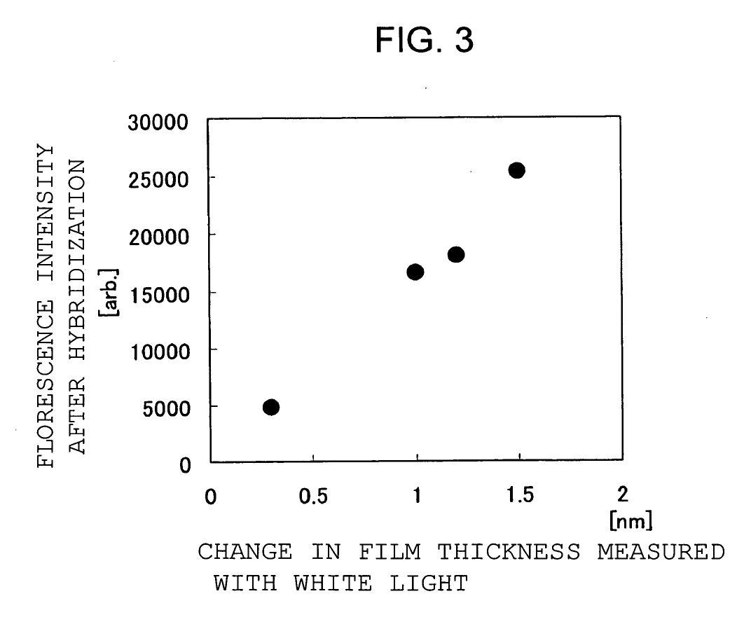

For the comparison with the thus-calculated hybridization amount, hybridization is performed by use of the target DNA on which the fluorescence molecule is modified, as a general method, and the hybridization amount is calculated by use of the fluorescence scanner. Since a reagent Cys is employed as a fluorescence molecule, which is manufactured by Amersham Biosciences Corp, a laser of 635 nm is used as an exciting light, and laser scanning is performed on the slide glass. The fluorescence light thus obtained is detected with a space resolving power of 10 μm. There is found a correlation between the hybridization amount obtained from the fluorescence intensity and the hybridization amount obtained from the film thickness difference with the white light. The result thereof is shown in FIG. 3.

In the case of a conventional method which detects a fluorescence amount, a dynamic range available for the measurement is small. Therefore, if there are many spots on the slide glass, for example, it is difficult to measure the fluorescence amount while maintaining the measurement conditions of the fluorescence scanner unchanged. Consequently, by adjusting the sensitivity of the detector, the dynamic range is expanded. For example, if a voltage applied to a photoelectron multiplier of the detecting system and the laser intensity of the excited light are changed, detection of all of the spots is possible. However, if those measuring conditions vary depending on the spot, it is difficult to compare all the spots quantitatively.

On the other hand, when a film thickness is measured by use of the white light, the absolute film thickness can be obtained with respect to all of the spots. Therefore, there is an advantage that quantitative comparison is possible as to all of the spots, or between biochips.

In the present embodiment, a halogen lamp was used as a white light source. However, a discharge lamp such as a mercury lamp or a metal halide lamp, or a white LED, may be applicable. In the present embodiment, DNA was employed as a biomolecule. However, similar results can be obtained if another biomolecule is employed, such as RNA, protein, PNA, sugar chain, and a composite of those elements. In addition, with the method according to the present embodiment, a biomolecule was detected. However, a biochemical reaction may also be detected.

A similar testing and detection can be conducted, when quartz, plastics, metallic coating substrate or the like, besides the slide glass, is used as a substrate, in any size or shape thereof. In the present embodiment, the diameter of the spotted probe DNAs is around 300 μm, but even for another spot size, similar testing and detection can be conducted.

While we have shown and described several embodiments in accordance with our invention, it should be understood that disclosed embodiments are susceptible of changes and modifications without departing from the scope of the invention. Therefore, we do not intend to be bound by the details shown and described herein but intend to cover all such changes and modifications that fall within the ambit of the appended claims.

What is claimed is:

1. A biomolecule thin film measuring method having a process for detecting a biomolecule, by use of a biosensor element with probe biomolecules immobilized on a substrate, comprising,
1) a step which mounts on a stage, a biosensor element where the probe biomolecules are immobilized,
2) a step which irradiates said biosensor element with a white light,
3) a step which detects an interference fringe generated by allowing a reflected light from said biosensor element to interfere with a reflected light from a reference plane,
4) a step which obtains either of a distance and an optical path length between said biosensor element and a source of the white light, either of which maximizes a modulation amount of the interference fringe,
5) a step which calculates a three-dimensional shape of the surface of said biosensor element, from either of said distance and said optical path length,
6) a step which obtains height T1 of a part where the probe biomolecules are immobilized, from the three-dimensional shape thus calculated,
7) a step which allows said biosensor element to react with a solution containing a biomolecule,
8) a step which performs all the steps 1) to 5) as described above, for said biosensor element which has been subjected to the reaction,
9) a step which obtains height T2 of a part where the probe biomolecules are immobilized, from the three-dimensional shape thus calculated, and
10) a step which calculates a difference (T2−T1), between T2 obtained in step 9) and T1 obtained in step 6).

2. A method for manufacturing a biosensor element, having probe biomolecules immobilized on a substrate, comprising,
1) a step which mounts on a stage, a biosensor element having the probe biomolecules being immobilized,
2) a step which irradiates either of said biosensor element with a white light,
3) a step which detects an interference fringe generated by allowing a reflected light from said biosensor element to interfere with a reflected light from a reference plane,
4) a step which obtains either of a distance and an optical path length between said biosensor element, and a source of said white light, either of which maximizes a modulation amount of the interference fringe,
5) a step which calculates a three-dimensional shape of the surface of said biosensor element, from either of said distance and said optical path length,
6) a step which obtains an average height T1 on a part where the probe biomolecules are immobilized, and height variations Cv1, and
7) a step which conducts quality control of said probe biomolecules with thus obtained T1 and height variations Cv1.

3. A method for manufacturing a biosensor element, having probe biomolecules immobilized on a substrate, comprising,
1) a step which mounts on a stage, a biosensor substrate in a state prior to having the probe biomolecules being immobilized,
2) a step which irradiates said substrate with a white light,
3) a step which detects an interference fringe generated by allowing a reflected light from said substrate to interfere with a reflected light from a reference plane,
4) a step which obtains either of a distance and an optical path length between either of the biosensor element and the substrate, and a source of the white light, either of which maximizes a modulation amount of the interference fringe,
5) a step which calculates a three-dimensional shape of the surface of said substrate, from either of said distance and said optical path length,
6) a step which obtains surface variations Cv1, from thus obtained three-dimensional shape, and
7) a step which conducts quality control of said probe biomolecules with thus obtained variations C1.
